(19) United States
(12) Patent Application Publication
(10) Pub. No.: US 2016/0296387 A1
(43) Pub. Date: Oct. 13, 2016

(54) TRANSFER DEVICE FOR A STRETCHER

(71) Applicant: SCHÄR ENGINEERING AG, Flaach (CH)

(72) Inventor: Hugo Schar, Niederwil (CH)

(73) Assignee: Schaer Proton AG, Flaach (CH)

(21) Appl. No.: 15/100,540

(22) PCT Filed: Nov. 14, 2014
(86) PCT No.: PCT/CH2014/000164

§ 371 Date: May 31, 2016

(30) Foreign Application Priority Data
Dec. 4, 2013 (CH) 02003/13

(51) Int. Cl. A61G 1/00; A61G 1/02; A61B 6/04; A61G 1/048

(52) U.S. Cl. CPC A61G 1/00; A61G 1/048; A61G 1/02; A61B 6/0407; A61N 2005/1063

(57) ABSTRACT

Disclosed is a stretcher (3) that is removably connected to a transport device (1) in order to be transported. The positioning and coupling parts for securing the stretcher (3) to the therapy table (25) and the examination table are integrated into a space (32) inside the stretcher body (13), so the stretcher body (13) can be placed directly on the surface of the therapy table (25) and can be positioned thereon and secured thereto.

### BACKGROUND

The subject matter of the present invention relates to a transfer device for a stretcher for radio oncology.

Transfer devices for stretchers are used when patients must be radiated for tumor therapy. In particular in proton radiation, an optimal positioning of the patient in the radiation machine is of the utmost importance in order to allow aligning the effective range of the proton radiation precisely to the tumor and impact it there. For a precise determination of the location of a tumor, prior to radiation the exact position of the tumor in the body must be determined in a diagnostics center using a diagnostics machine.

Due to the fact that the position and the shape of the tumor in the body of the patient is influenced by the type of positioning of the patient it is beneficial to place the patient already in the diagnostics machine in the same form or fixation (e.g., cushions adjustable to the body, which are rigid when a vacuum is applied), as required in the therapy machine. Ideally, in diagnostics the same forms and/or supporting means are used for the patient as also applied during the therapy. This results in the desire to use the same stretches accepting the forms, both in diagnostics as well as in therapy. Professional language calls these mobile stretchers a “treatment couch”.

This way, these stretches can be already used in separate preparation rooms, i.e. the patient can be prepared in the respective form on the stretcher outside the diagnostics area or the therapy area free from stress and this way the duration of the patient in the diagnostics area and the therapy rooms at the respective machines can be considerably shortened.

At the therapy machines the patients are radiated from the top and from the side. Additionally, in a limited area of the stretcher, radiation is also required from the bottom through said stretcher, i.e. in this area radiation is possible by 360° around the patient. This zone shows a length of approx. 1-1.3 m. In this area the stretcher must be designed such that the radiation can occur through the stretcher and can be calculated with regards to its effect upon the tumor. This means, no coupling parts, screws, etc. may be fastened here, which provide for a stable and precise coupling to the patient positioning systems at the therapy machines or allow a positioning on the examination tables at the diagnostics machines.

The diagnostics machines exhibit individual patient positioning systems, which represent an integral component of the certified product, e.g., CT, MRI, or CT-PET. Ideally it should be possible to place the stretchers without any distance upon the patient positioning systems at the diagnostics machine. This goal has been attained in prior art with so-called sliding couches, which are pushed from a transport cart to the diagnostics and/or therapy machines, or which are coupled to the therapy machine on the bottom of the couch.

One characteristic of these solutions is the fact that during the displacement of the stretchers the couches have to be supported longitudinally at their bottom during the displacement of the stretchers between the preparation, diagnostics, and therapy rooms. This is particularly difficult if the stretchers in the area, which can also be radiated from the bottom, exhibit recesses and/or are shortened in order to allow direct radiation from the bottom.

The stretchers exhibit a thickness or height of approx. 5 cm. This way, the patient is vertically shifted upwards by 5 cm during use in the diagnostics machine. When additional, projecting coupling elements are applied at the stretchers and the counterparts of said coupling elements are applied on the diagnostics machine, here the patient is shifted upwards by another distance (approx. 4 cm or more). This way, patients of a slightly larger size no longer rest in the area in which the diagnostics machines can generate high-quality images.

### SUMMARY

One objective of the invention is to generate a transfer device which corrects the disadvantages and shortcomings of the solutions of prior art and which allows to position the patients optimally on the therapy table.

This objective is attained in a transfer device for a stretcher according to the invention. Advantageous embodiments of the transfer device are described below and in the claims.

By the arrangement of the clamping elements inside the flat body of the stretcher, measuring only a few centimeters in thickness, it is possible to place the stretcher directly upon the therapy table and thus considerably reduce the structural height at the diagnostics machine in reference to stretchers of prior art and/or limit it to the thickness of the stretcher and simultaneously improve essentially the use of imagery of the diagnostics machines.

In order to use these opportunities without limitations, the coupling of the stretcher for fastening the stretcher at the transport cart is mounted at the opposite end of the therapy area of the stretcher, rendering the bottom of the stretcher essentially free from disturbing metal parts over its entire length and thus free over its entire area for the contact with the diagnostics machines or the therapy machines. When the patient positioning system engages the bottom of the stretcher, the patient positioning system moves slightly higher than necessary. This way it is achieved that the stretcher rests smoothly and, if the coupling was not precisely positioned in the horizontal direction, it also ensures that no tipping occurs of the stretcher. The vertical motion is executed by the positioning system, i.e. the cart, which carries the stretcher, thus it shows only a vertical guidance without any drive for the coupling. After the short vertical motion the stretcher can be released from the coupling at the transport cart. Any unavoidable height differences between the stretcher seat at the transport cart and the surfaces at the tables are compensated. The transfer from the diagnostics machine to the cart occurs in the inverse sequence. As soon as the stretcher is accepted by the cart and the patient positioning system has lowered, the stretcher also lowers slightly until it rests on a mechanical stop at the coupling in the transport cart.

In the area of the stretcher not subject to radiation, coupling parts may be located inside the stretcher, which allow a horizontal adjustment and positioning of the stretcher on the examination and therapy tables. The centering mechanism (passive centering) is integrated inside the stretcher and oriented in the coupling parts (active centering) provided at the patient tables, which project into the stretcher when the patient table is raised. Of course, the active centering elements may also be arranged at the stretcher (preferably inside the thickness of the stretcher) and passive centering and/or clamping elements may be provided at the tables.

### BRIEF DESCRIPTION OF THE DRAWINGS

Based on the illustrated exemplary embodiment the invention is explained in greater detail. It shows:

FIG. 1 a transport cart with a coupled stretcher,

FIG. 2 a transport cart in front of a diagnostics machine,

FIG. 3 a transport cart at the diagnostics machine shortly before placement of the stretcher onto the therapy table,

FIG. 4 a longitudinal section through the stretcher,

FIG. 5 a top view of the stretcher with a clamping means integrated in the stretcher body,

FIG. 6 a side view of the stretcher shortly before placement on the positioning system at the therapy machine, and

FIG. 7 a top view of the stretcher and the positioning system, the latter shown in dot-dash lines.

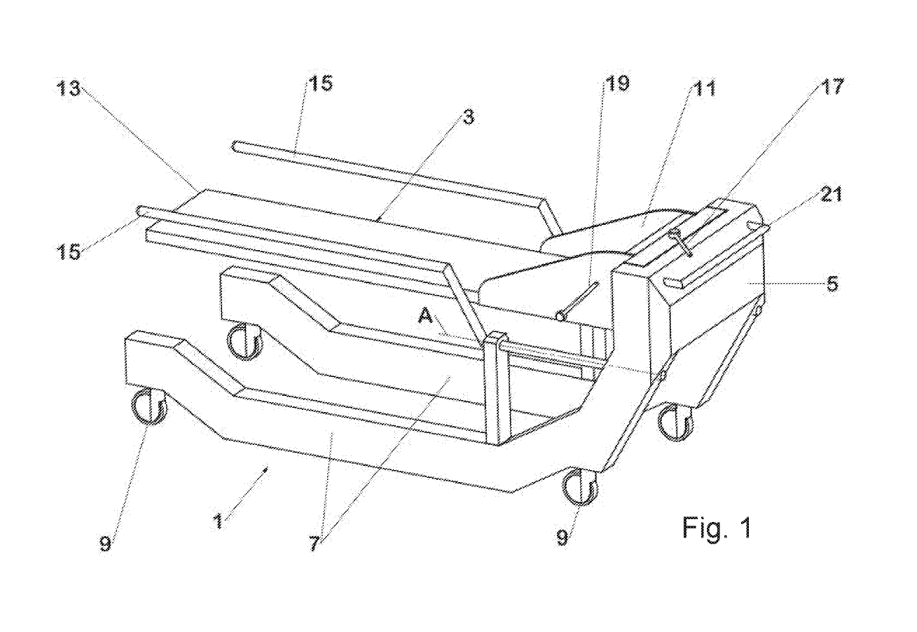

### DETAILED DESCRIPTION OF THE PREFERRED EMBODIMENTS

The reference character 1 marks a transport cart for a stretcher 3, also called a treatment couch. The transport cart 1 comprises for example a head part 5 with a vertical guide for a coupling device. Two projections 7, forming a chassis and located laterally at a distance are provided at the bottom of the head part 5, with two wheels 9 each being arranged thereat. The vertical guide, not driven, and a coupling device are installed in the head part 5 (the vertical guide and the coupling device are not shown). The coupling device serves to carry a coupling head 11, which is detachably connected to the stretcher 3 and/or the plate-shaped therapy body 13 forming the stretcher 3. The coupling elements at the coupling head 11 for the detachable connection to the stretcher are not shown either.

Additionally, two fastening rods 15 may be fastened at the transport cart 1, which are supported in a manner pivotally downwards about an axis A. The coupling of the stretcher 3 occurs at the vertical guide 11 via a lever 17. The releasing of the connection coupling between the vertical guide in the head part 5 and the coupling head 11 occurs also with the lever 17. The transport cart can be pushed manually at a guidance rod 21 or, if so desired, displaced sensor-controlled by an electric drive of the wheels 9.

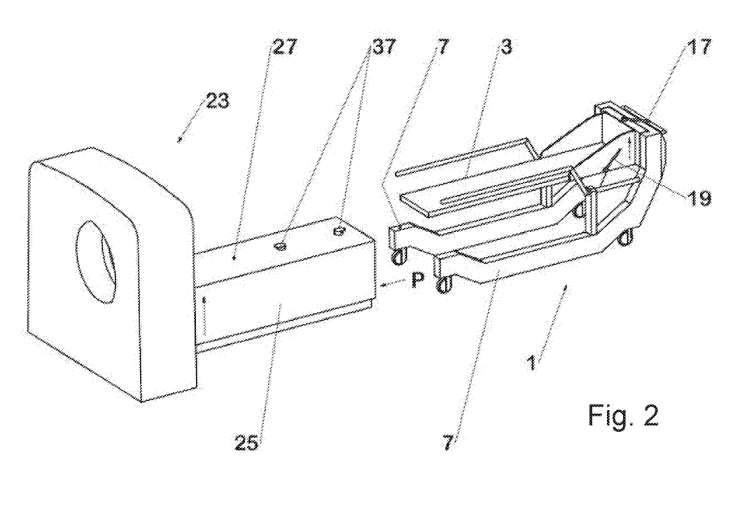

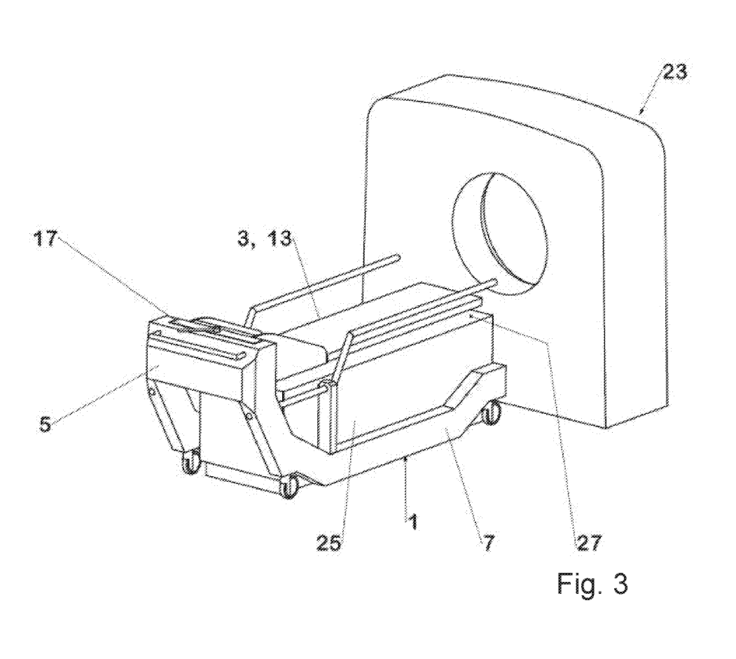

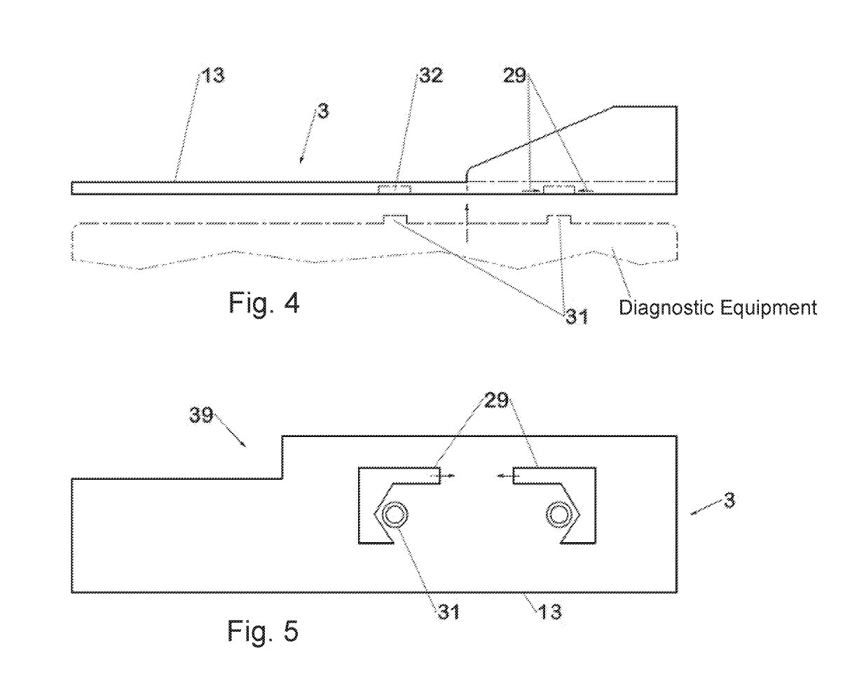

The distance of the two cantilevers 7 is selected such that the stretcher 3 comes to rest during the approaching of the transport cart 1 to a diagnostics machine 23 and/or to its therapy table 25 above said table 25 (FIGS. 2 and 3). When the transport cart 1 is moved in the direction of the arrow P (FIG. 2) over the therapy table 25 the bottom of the stretcher 3 is positioned slightly above the surface 27 of the therapy table 25. When the end position is approximately reached (FIG. 3) the therapy table 25 is raised. By this lifting motion the stretcher 3 is slightly raised, which is possible by the parallel guidance in the head part 5 without here the stretcher 3 being decoupled from the head part 5. By operating the two levers 19 the stretcher 3 is centered on the surface of the therapy table 25 in the horizontal position. The centering mechanism 29 is installed between the two surfaces of the stretcher, i.e. the surface contacting at the top and the surface located at the bottom, inside the stretcher 3 and is oriented in the coupling parts fastened on the patient table 25, e.g., cylindrical pins 31, which upon raising the therapy table 5 project from the bottom into a space 32 at the bottom of the stretcher 3. Passive coupling parts 35, e.g., sections with undercuts, are formed in the bottom and/or inside the stretcher 3, allowing actively mobile coupling parts 37 to engage them and e.g., the passive coupling parts 35 engage therebehind by a spreading motion. By the spreading motion of the active coupling parts 37, which are arranged at the therapy table 25, not only a fastening of the stretcher 3 occurs on the therapy table 25, but in advance or simultaneously also an absolutely precise, reproducible positioning of the stretcher 3 in the horizontal level. Therefore the two coupling parts 35 and 37 form a positioning system 33 for the stretcher 3. The passive and active coupling parts 35, 37 are preferably made from non-magnetic materials, allowing the stretcher 3 to be used also in MRIs, for example. With the embodiment and arrangement of the coupling parts 35, 37 according to the invention, here recesses 39 can also be embodied at the stretcher 3, which grant to the patient resting on the stretcher 3 optimal access to the radiation machine. After placing the couch/stretcher 3 on the table 25 at the diagnostics machine or the coupling of the couch to the patient positioning system at the therapy machine, by operating the first lever 17, the stretcher 3 is separated from the head part 5 of the transport cart 1. The transport cart 1 can now be guided away from the therapy table 25.

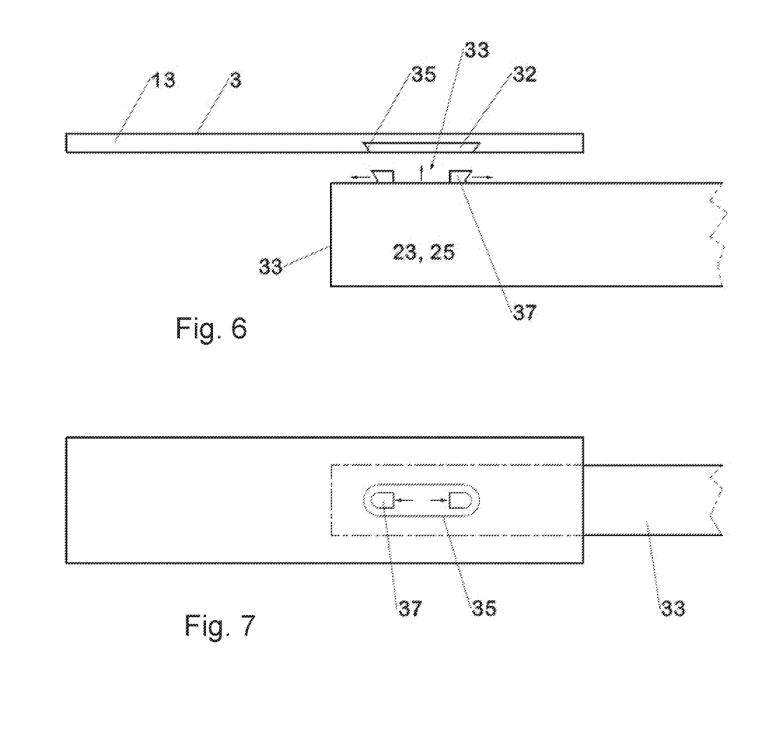

In the embodiment of the invention according to FIGS. 6 and 7 the passive coupling part 35 is embodied in the stretcher body 13 of the stretcher 3. In this embodiment the passive coupling part 35 shows an undercut in the space 32, allowing active coupling parts 37 to engage it, which are embodied at the table 23 of the diagnostics machine and at the table 25 of the therapy machine in a vertically displaceable and horizontally expandable fashion. The operation of the active coupling parts at the tables 23, 25 occurs by electro-mechanical or manually operated actuators, which are not shown in the figures.

1. A transfer device for a stretcher (3) comprising a stretcher and a transport cart (1) for transfer of the stretcher (3) from a first table at a diagnostics machine to a second table at a therapy machine for radiation treatment of a person resting on a stretcher (3), the stretcher (3) comprising a tightening element, by which the stretcher (3) is fastenable in a precisely positioned fashion at the tables of the diagnostics and therapy machines, the stretcher (3) comprises a flat, plate-shaped stretcher body (13), with a space (32) being formed between surfaces thereof for inserting a coupling part (37, 31) from a bottom through a lower surface of the stretcher body (13) and for positioning and for fastening the stretcher (3) on the tables (23, 25).

2. The transfer device according to claim 1, wherein the space (32) forms a passive coupling part (35), which is engagable from the bottom by an active coupling part (37) and which is inserted for the purpose of positioning and fastening.

3. The transfer device according to claim 1, wherein the space (32) is embodied such that an active coupling part (29) is located therein, which engages a passive coupling part (31) that is inserted from the bottom for the purpose of positioning and fastening.

4. The transfer device according to claim 1, wherein the stretcher (3) comprises a head part (5) by which the stretcher (3) is fastened and supported at the head part of the transport cart (1) and which is mobile in a passive vertical fashion for decoupling.
